(19) United States
(12) Patent Application Publication
(10) Pub. No.: US 2009/0280104 A1
(43) Pub. Date: Nov. 12, 2009

(54) COMPOSITIONS AND METHODS FOR INHIBITING SHIGA TOXIN AND SHIGA-LIKE TOXIN

(75) Inventors: Jan Holgersson, Huddinge (SE); Anki Gustafsson, Johannesshov (SE)

Correspondence Address: MINTZ, LEVIN, COHN, FERRIS, GLOVSKY AND POPEO, P.C, ONE FINANCIAL CENTER, BOSTON, MA, 02111, US

(73) Assignee: RECOPHARMA AB, Huddinge (SE)

(21) Appl. No.: 12/463,943

(22) Filed: May 11, 2009

Related U.S. Application Data
(60) Provisional application No. 61/051,874, filed on May 9, 2008.

(51) Int. Cl. A61K 38/45; C12N 9/10; C12P 21/06; C12N 5/10; C12N 1/16; C12N 1/21

(52) U.S. Cl. 424/94.5; 435/193; 435/69.7; 435/358; 435/254.2; 435/252.3

(57) ABSTRACT

The present invention provides compositions and methods for treating or preventing infection by shiga toxin producing bacteria.

### REFERENCE TO RELATED APPLICATIONS

This non-provisional application claims priority under 35 U.S.C. §119(e) to U.S. Provisional Application No. 61/051,874 filed May 9, 2008, the contents of which are hereby incorporated by reference in its entirety.

### FIELD OF THE INVENTION

The invention relates to generally to compositions and methods for treating or preventing infection by shiga toxin and shiga-like toxin producing bacteria and more particularly to compositions including fusion polypeptides comprising carbohydrate epitopes that inhibit shiga toxin and shiga-like toxins.

### BACKGROUND OF THE INVENTION

Shiga and shiga-like toxins consist of a toxic A subunit and five carbohydrate binding B subunits. Shiga toxin is produced by Shigella dysenteriae. The toxin binds to Gb3 (Galα4Galβ4Glcβ1Cer)-expressing cells and, upon internalization, inhibits protein synthesis leading to diarrhoea, hemorrhagic colitis or haemolytic uremic syndrome in infected individuals. It has been shown that cytokines induced by S. dysenteriae infection can cause production of Gb3 in some cells. Shiga-like toxin 1 is nearly identical with shiga toxin and also recognizes Gb3. Shiga-like toxin 2 exists in different forms, most of them also recognize Gb3 but one form has been shown to bind to Gb4 (GalNAcβ3Galα4Galβ4Glcβ1Cer) as well. Studies indicate that the lipid part of the carbohydrate ligand also plays an important role in recognition. Shiga-like toxin 1 and 2 are produced mainly by enterohaemorrhagic E. coli (EHEC), but also by Aeromononas caviae, Aeromononas hydrophila, Citrobacter freundii and Enterobacter cloacae. Despite the similarity between shiga toxins and shiga-like toxins, differences do exist with regard to effects on cells and interactions with the immune system of the host.

### SUMMARY OF THE INVENTION

The invention is based in part on the discovery that carbohydrate epitopes that mediate (i.e., block, inhibit) the binding of shiga and/or shiga-like toxin to a host cell surface can be specifically expressed at high density and by different core saccharide chains on mucin-type protein backbones. The polypeptides are referred to herein as shiga toxin inhibitin (STI) fusion proteins or SI polypeptides. These recombinant, heavily glycosylated proteins carrying ample O-linked glycans capped with carbohydrate determinants with known bacterial toxin-binding activity can act as decoys, and as such specifically prevent (e.g., sterically inhibit) bacterial toxin infection in for example, the respiratory or the gastrointestinal tracts. The fusion proteins have low toxicity and low risk of inducing bacterial resistance to the drugs.

In one aspect, the invention provides a fusion polypeptide that includes a first polypeptide that carries the Galα4Galβ3GalNAcα and/or Galα4Galβ4GlcNac carbohydrate epitope, operably linked to a second polypeptide. The first polypeptide is multivalent for these epitopes. The first polypeptide is, for example, a mucin polypeptide such as PSGL-1 or portion thereof. Preferably, the mucin polypeptide is the extracellular portion of PSGL-1.

The second polypeptide comprises at least a region of an immunoglobulin polypeptide. For example, the second polypeptide comprises a region of a heavy chain immunoglobulin polypeptide. Alternatively, the second polypeptide comprises the Fc region of an immunoglobulin heavy chain.

The fusion polypeptide is a multimer. Preferably, the fusion polypeptide is a dimer.

Also included in the invention is a nucleic acid encoding the SI fusion polypeptide, as well as a vector containing SI fusion polypeptide-encoding nucleic acids described herein, and a cell containing the vectors or nucleic acids described herein. Optionally, the vector further comprises a nucleic acid encoding one or more glycosyltransferases necessary for the synthesis of the desired carbohydrate epitope. For example, the vector contains a nucleic acid encoding a α1,4-galactosyltransferase, and optionally a nucleic acid encoding a core 2β1,6-N-acetylglucosaminyltransferase.

In another aspect, the invention provides a method of inhibiting (e.g., decreasing) the binding of shiga toxin and/or shiga-like toxin to a cell surface. Binding is inhibited by contacting shiga and or shiga-like toxin producing bacteria or free shiga and shiga-like toxin with the SI fusion polypeptide. The invention also features methods of preventing or alleviating a symptom of shiga and/or shiga-like toxin producing bacterial infection or a disorder associated with shiga and/or shiga-like toxin producing bacterial infection in a subject by identifying a subject suffering from or at risk of developing shiga and/or shiga-like toxin producing bacterial infection and administering to the subject the fusion polypeptide of the invention. The bacteria is for example, Shigella dystenteriae (S. dysenteriae), Escheria Coli (E. Coli), enterohaemorrhagic E. Coli, Aeromononas caviae (A. Caviae), Aeromononas hydrophila (A. hydrophila), Citrobacter freundii (C. freundii) and Enterobacter cloacae (E. cloacae).

The subject is a mammal such as human, a primate, mouse, rat, dog, cat, cow, horse, pig. The subject is suffering from or at risk of developing a shiga and/or shiga-like toxin producing bacterial infection or a disorder associated with a shiga and/or shiga-like toxin producing bacterial infection. A subject suffering from or at risk of developing a shiga and/or shiga-like toxin producing bacterial infection or a disorder associated with a shiga and/or shiga-like toxin producing bacterial infection is identified by methods known in the art

Also included in the invention are pharmaceutical compositions that include the fusion polypeptides of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

The invention is based in part in the discovery that carbohydrate epitopes that mediate (i.e., block, inhibit) the binding activity of shiga and/or shiga-like toxin can be specifically expressed at high density on glycoproteins, e.g., mucin-type protein backbones. This higher density of carbohydrate epitopes results in an increased valancy and affinity compared to monovalent oligosaccharides and wild-type, e.g. native non recombinantly expressed glycoproteins.

Shiga toxin and shiga-like toxin producing bacteria bind to host cells via specific cell surface glycoplipids, Gb3 (Galα4Galβ4Glcβ1Cer) and/or Gb4 (GalNAcβ3Galα4Galβ4Glcβ1Cer). Upon binding to the surface of a host cell, the toxin is internalized and causes inhibition of protein synthesis within target cells. After entering the cell, the protein functions as an N-glycosidase, cleaving several nucleobases from the RNA that comprises the ribosome, thereby halting protein synthesis, resulting in diarrhea, hemorrhagic colitis and/or hemolytic uremic syndrome.

The invention provides glycoprotein-immunoglobulin fusion proteins (refered to herein as “SI fusion protein or SI fusion peptides”) containing multiple Galα4Galβ3GalNAcα and/or Galα4Galβ4GlcNAc epitopes, that are useful in mediating (i.e., blocking, inhibiting) the binding interaction between shiga toxin and/or shiga-like toxin and a host cell surface. The epitopes are terminal, i.e, at the terminus of the glycan. The SI fusion protein inhibits 10%, 20%, 30%, 40%, 50%, 60%, 70%, 80%, 90%, 95%, 98% or 100% of the binding of a shiga toxin and/or shiga-like toxin to a cell surface. For example, the SI fusion proteins are useful in inhibiting the binding of shiga toxin, shiga-like toxin 1 and/or shiga-like toxin 2 to host cell surfaces.

The SI fusion peptide is more efficient on a carbohydrate molar basis in the binding activity of inhibiting shiga and/or shiga-like toxin as compared to free saccharrides. The SI fusion peptide inhibits 2, 4, 10, 20, 50, 80, 100 or more-fold greater amount of toxin as compared to an equivalent amount of free saccharrides.

### Fusion Polypeptides

In various aspects the invention provides fusion proteins that include a first polypeptide containing at least a portion of a glycoprotein, e.g. a mucin polypeptide operatively linked to a second polypeptide. As used herein, a “fusion protein” or “chimeric protein” includes at least a portion of a glycoprotein polypeptide operatively linked to a non-mucin polypeptide.

A “mucin polypeptide” refers to a polypeptide having a mucin domain. The mucin polypeptide has one, two, three, five, ten, twenty or more mucin domains. The mucin polypeptide is any glycoprotein characterized by an amino acid sequence substituted with O-glycans. For example, a mucin polypeptide has every second or third amino acid being a serine or threonine. The mucin polypeptide is a secreted protein. Alternatively, the mucin polypeptide is a cell surface protein.

Mucin domains are rich in the amino acids threonine, serine and proline, where the oligosaccharides are linked via N-acetylgalactosamine to the hydroxy amino acids (O-glycans). A mucin domain comprises or alternatively consists of an O-linked glycosylation site. A mucin domain has 1, 2, 3, 5, 10, 20, 50, 100 or more O-linked glycosylation sites. Alternatively, the mucin domain comprises an N-linked glycosylation site. A mucin polypeptide has 50%, 60%, 80%, 90%, 95% or 100% of its mass due to the glycan. A mucin polypeptide is any polypeptide encode for by a MUC genes (i.e., MUC1, MUC2, MUC3, MUC4, MUC5a, MUC5b, MUC5c, MUC6, MUC11, MUC12, etc.). Alternatively, a mucin polypeptide is P-selectin glycoprotein ligand 1 ( PSGL-1), CD34, CD43, CD45, CD96, GlyCAM-1, MAdCAM-1, red blood cell glycophorins, glycocalicin, glycophorin, sialophorin, leukosialin, LDL-R, ZP3, and epiglycanin. Preferably, the mucin is PSGL-1. PSGL-1 is a homodimeric glycoprotein with two disulfide-bonded 120 kDa subunits of type 1 transmembrane topology, each containing 402 amino acids. In the extracellular domain there are 15 repeats of a 10-amino acid consensus sequence that contains 3 or 4 potential sites for addition of O-linked oligosaccharides. In one embodiment, the 10-amino acid consensus sequence is A(I) Q T T Q(PAR) P(LT) A(TEV) A(PG) T(ML) E (SEQ ID NO: 1). In another embodiment, the 10-amino acid consensus sequence is A Q(M) T T P(Q) P(LT) A A(PG) T(M) E (SEQ ID NO: 2). PSGL-1 is predicted to have more than 53 sites for O-linked glycosylation and 3 sites for N-linked glycosylation in each monomer.

The mucin polypeptide contains all or a portion of the mucin protein. Alternatively, the mucin protein includes the extracellular portion of the polypeptide. For example, the mucin polypeptide includes the extracellular portion of PSGL-1 or a portion thereof (e.g., amino acids 19-319 disclosed in GenBank Accession No. A57468). The mucin polypeptide also includes the signal sequence portion of PSGL-1 (e.g., amino acids 1-18), the transmembrane domain (e.g., amino acids 320-343), and the cytoplamic domain (e.g., amino acids 344-412).

A “non-mucin polypeptide” refers to a polypeptide of which at least less than 40% of its mass is due to glycans.

Within an SI fusion protein of the invention the mucin polypeptide corresponds to all or a portion of a mucin protein. An SI fusion protein comprises at least a portion of a mucin protein. “At least a portion” is meant that the mucin polypeptide contains at least one mucin domain (e.g., an O-linked glycosylation site). The mucin protein comprises the extracellular portion of the polypeptide. For example, the mucin polypeptide comprises the extracellular portion of PSGL-1.

The first polypeptide is glycosylated by one or more glycosyltransferases. The first polypeptide is glycosylated by 2, 3, 5 or more glycosyltransferases. Glycosylation is sequential or consecutive. Alternatively glycosylation is concurrent or random, i.e., in no particular order. The first polypeptide is glycosylated by any enzyme capable of adding or producing N-linked or O-linked glycans to or on a protein backbone. For example the first polypeptide is glycosylated by α1,4 galactosyltransferase. Suitable sources for α1,4 galactosyltransferase include but are not limited to GenBank Accession Nos. NP_{—}059132, AAO39150, ABP35533, ABP35532, ABQ10741, ABQ10740, AAS77221, AAS77220, AAS77219, AAS77216, AAS77215, AAS77214, AAX20109, AA039151, AAO39149, AAP47170, AAP47169, AAP47168, AAP47167, AAP47166, AAP47165, and AAP47164, and are incorporated herein by reference in their entirety. In a particular embodiment, the first polypeptide is glycosylated by both α1,4 galactosyltransferase and core 2β1,6-N-acetylglucosaminyltransferase. Suitable sources for core 2β1,6-N-acetylglucosaminyltransferase include but are not limited to GenBank Accession Nos. CAA79610, Z19550, BAB66024, AP001515, AJ420416.1, AK313343.1, AL832647.2, AY196293.1, BC074885.2, BC074886, BC109101, BC109102.1, M97347.1, BAG36146.1, CAD89956.1, AAH74885.1, AAH74886.1, AAI09102.1, AAI09103.1, AAA35919.1, AAH17032, 095395, NP_{—}004742, EAW77572, NP_{—}004742.1, BC017032, AF102542.1, AAD10824.1, AF038650.1, NM_{—}004751.2, Q9P109, NP_{—}057675, EAW95751, AF132035.1, AAF63156.1, and NP_{—}057675.1. The first polypeptide contains greater than 40%, 50%, 60%, 70%, 80%, 90% or 95% of its mass due to carbohydrate.

Within the fusion protein, the term “operatively linked” is intended to indicate that the first and second polypeptides are chemically linked (most typically via a covalent bond such as a peptide bond) in a manner that allows for O-linked and/or N-linked glycosylation of the first polypeptide. When used to refer to nucleic acids encoding a fusion polypeptide, the term operatively linked means that a nucleic acid encoding the mucin polypeptide and the non-mucin polypeptide are fused in-frame to each other. The non-mucin polypeptide can be fused to the N-terminus or C-terminus of the mucin polypeptide.

The SI fusion protein is linked to one or more additional moieties. For example, the SI fusion protein may additionally be linked to a GST fusion protein in which the SI fusion protein sequences are fused to the C-terminus of the GST (i.e., glutathione S-transferase) sequences. Such fusion proteins can facilitate the purification of the SI fusion protein. Alternatively, the SI fusion protein may additionally be linked to a solid support. Various solid supports are known to those skilled in the art. For example, the SI fusion protein is linked to a particle made of, e.g. metal compounds, silica, latex, polymeric material; a microtiter plate; nitrocellulose, or nylon or a combination thereof. The SI fusion proteins linked to a solid support can be used as a diagnostic or screening tool for infections caused by shiga toxin and shiga-like toxin producing bacteria.

The fusion protein includes a heterologous signal sequence (i.e., a polypeptide sequence that is not present in a polypeptide encoded by a mucin nucleic acid) at its N-terminus. For example, the native mucin glycoprotein signal sequence can be removed and replaced with a signal sequence from another protein. In certain host cells (e.g., mammalian host cells), expression and/or secretion of polypeptide can be increased through use of a heterologous signal sequence.

A chimeric or fusion protein of the invention can be produced by standard recombinant DNA techniques. For example, DNA fragments coding for the different polypeptide sequences are ligated together in-frame in accordance with conventional techniques, e.g. by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene is synthesized by conventional techniques including automated DNA synthesizers. Alternatively, PCR amplification of gene fragments is carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that encode a fusion moiety (e.g., an Fc region of an immunoglobulin heavy chain). A mucin encoding nucleic acid can be cloned into such an expression vector such that the fusion moiety is linked in-frame to the immunoglobulin protein.

SI fusion polypeptides may exist as oligomers, such as dimers, trimers or pentamers. Preferably, the SI fusion polypeptide is a dimer.

The first polypeptide, and/or nucleic acids encoding the first polypeptide, is constructed using mucin encoding sequences are known in the art. Suitable sources for mucin polypeptides and nucleic acids encoding mucin polypeptides include GenBank Accession Nos. NP663625 and NM145650, CAD10625 and AJ417815, XP140694 and XM140694, XP006867 and XM006867 and NP00331777 and NM009151 respectively, and are incorporated herein by reference in their entirety.

The mucin polypeptide moiety is provided as a variant mucin polypeptide having an alteration in the naturally-occurring mucin sequence (wild type) that results in increased carbohydrate content (relative to the non-mutated sequence). As used herein, an alteration in the naturally-occurring (wild type) mucin sequence includes one or more one or more substitutions, additions or deletions into the nucleotide and/or amino acid sequence such that one or more amino acid substitutions, additions or deletions are introduced into the encoded protein. Alterations can be introduced into the naturally-occurring mucin sequence by standard techniques, such as site-directed mutagenesis and PCR-mediated mutagenesis.

For example, the variant mucin polypeptide comprised additional O-linked glycosylation sites compared to the wild-type mucin. Alternatively, the variant mucin polypeptide comprises an amino acid sequence alteration that results in an increased number of serine, threonine or proline residues as compared to a wild type mucin polypeptide. This increased carbohydrate content can be assessed by determining the protein to carbohydrate ratio of the mucin by methods known to those skilled in the art.

Alternatively, the mucin polypeptide moiety is provided as a variant mucin polypeptide having alterations in the naturally-occurring mucin sequence (wild type) that results in a mucin sequence with more O-glycosylation sites or a mucin sequence preferably recognized by peptide N-acetylgalactosaminyltransferases resulting in a higher degree of glycosylation.

In some embodiments, the mucin polypeptide moiety is provided as a variant mucin polypeptide having alterations in the naturally-occurring mucin sequence (wild type) that results in a mucin sequence more resistant to proteolysis (relative to the non-mutated sequence).

The first polypeptide includes full-length PSGL-1. Alternatively, the first polypeptide comprise less than full-length PSGL-1 polypeptide, e.g., a functional fragment of a PSGL-1 polypeptide. For example the first polypeptide is less than 400 contiguous amino acids in length of a PSGL-1 polypeptide, e.g., less than or equal to 300, 250, 150, 100, or 50, contiguous amino acids in length of a PSGL-1 polypeptide, and at least 25 contiguous amino acids in length of a PSGL-1 polypeptide. The first polypeptide is, for example, the extracellular portion of PSGL-1, or includes a portion thereof Exemplary PSGL-1 polypeptide and nucleic acid sequences include GenBank Access No: XP006867; XM006867; XP140694 and XM140694.

The second polypeptide is preferably soluble. In some embodiments, the second polypeptide includes a sequence that facilitates association of the SI fusion polypeptide with a second mucin polypeptide. The second polypeptide includes at least a region of an immunoglobulin polypeptide. “At least a region” is meant to include any portion of an immunoglobulin molecule, such as the light chain, heavy chain, Fc region, Fab region, Fv region or any fragment thereof. Immunoglobulin fusion polypeptide are known in the art and are described in e.g. U.S. Pat. Nos. 5,516,964; 5,225,538; 5,428,130; 5,514,582; 5,714,147; and 5,455,165.

The second polypeptide comprises a full-length immunoglobulin polypeptide. Alternatively, the second polypeptide comprises less than full-length immunoglobulin polypeptide, e.g. a heavy chain, light chain, Fab, Fab₂, Fv, or Fc. Preferably, the second polypeptide includes the heavy chain of an immunoglobulin polypeptide. More preferably the second polypeptide includes the Fc region of an immunoglobulin polypeptide.

The second polypeptide has less effector function than the effector function of an Fc region of a wild-type immunoglobulin heavy chain. Alternatively, the second polypeptide has similar or greater effector function of an Fc region of a wild-type immunoglobulin heavy chain. An Fc effector function includes for example, Fc receptor binding, complement fixation and T cell depleting activity (see for example, U.S. Pat. No. 6,136,310). Methods of assaying T cell depleting activity, Fc effector function, and antibody stability are known in the art. In one embodiment the second polypeptide has low or no affinity for the Fc receptor. Alternatively, the second polypeptide has low or no affinity for complement protein Clq.

Another aspect of the invention pertains to vectors, preferably expression vectors, containing a nucleic acid encoding mucin polypeptides, or derivatives, fragments, analogs or homologs thereof. The vector contains a nucleic acid encoding a mucin polypeptide operably linked to a nucleic acid encoding an immunoglobulin polypeptide, or derivatives, fragments analogs or homologs thereof. Additionally, the vector comprises a nucleic acid encoding a glycosyltransferase such as an α1,4galactosyltransferase. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., SI fusion polypeptides, mutant forms of SI fusion polypeptides, etc.).

The recombinant expression vectors of the invention can be designed for expression of SI fusion polypeptides in prokaryotic or eukaryotic cells. For example, SI fusion polypeptides can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g. Gottesman, GENE EXPRFSSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

The SI fusion polypeptide expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, SI fusion polypeptide can be expressed in insect cells using baculovirus expression vectors. Baculovirus vectors available for expression of proteins in cultured insect cells (e.g., Mamestra brassicae cells or SF9 cells) include the pAc series (Smith, et al., 1983. Mol. Cell. Biol. 3: 2156-2165) and the pVL series (Lucklow and Summers, 1989. Virology 170: 31-39).

A nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, SI fusion polypeptides can be expressed in bacterial cells such as E. coli, insect cells such as M. brassicae, yeast or mammalian cells (such as human, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the fusion polypeptides or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) SI fusion polypeptides. Accordingly, the invention further provides methods for producing SI fusion polypeptides using the host cells of the invention. In one embodiment, the method comprises culturing the host cell of invention (into which a recombinant expression vector encoding SI fusion polypeptides has been introduced) in a suitable medium such that SI fusion polypeptides is produced. In another embodiment, the method further comprises isolating SI polypeptide from the medium or the host cell.

The SI fusion polypeptides may be isolated and purified in accordance with conventional conditions, such as extraction, precipitation, chromatography, affinity chromatography, electrophoresis or the like. For example, the immunoglobulin fusion proteins may be purified by passing a solution through a column which contains immobilized protein A or protein G which selectively binds the Fc portion of the fusion protein. See, for example, Reis, K. J., et al., J. Immunol. 132:3098-3102 (1984); PCT Application, Publication No. WO87/00329. The fusion polypeptide may then be eluted by treatment with a chaotropic salt or by elution with aqueous acetic acid (1 M).

Alternatively, SI fusion polypeptides according to the invention can be chemically synthesized using methods known in the art. Chemical synthesis of polypeptides is described in, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989). The polypeptides are purified so that they are substantially free of chemical precursors or other chemicals using standard peptide purification techniques. The language “substantially free of chemical precursors or other chemicals” includes preparations of peptide in which the peptide is separated from chemical precursors or other chemicals that are involved in the synthesis of the peptide. In one embodiment, the language “substantially free of chemical precursors or other chemicals” includes preparations of peptide having less than about 30% (by dry weight) of chemical precursors or non-peptide chemicals, more preferably less than about 20% chemical precursors or non-peptide chemicals, still more preferably less than about 10% chemical precursors or non-peptide chemicals, and most preferably less than about 5% chemical precursors or non-peptide chemicals.

Chemical synthesis of polypeptides facilitates the incorporation of modified or unnatural amino acids, including D-amino acids and other small organic molecules. Replacement of one or more L-amino acids in a peptide with the corresponding D-amino acid isoforms can be used to increase the resistance of peptides to enzymatic hydrolysis, and to enhance one or more properties of biologically active peptides, i.e., receptor binding, functional potency or duration of action. See, e.g. Doherty, et al., 1993. J. Med. Chem. 36: 2585-2594; Kirby, et al., 1993. J. Med. Chem. 36:3802-3808; Morita, et al., 1994. FEBS Lett. 353: 84-88; Wang, et al., 1993. Int. J. Pept. Protein Res. 42: 392-399; Fauchere and Thiunieau, 1992. Adv. Drug Res. 23: 127-159.

Introduction of covalent cross-links into a peptide sequence can conformationally and topographically constrain the polypeptide backbone. This strategy can be used to develop peptide analogs of the fusion polypeptides with increased potency, selectivity and stability. Because the conformational entropy of a cyclic peptide is lower than its linear counterpart, adoption of a specific conformation may occur with a smaller decrease in entropy for a cyclic analog than for an acyclic analog, thereby making the free energy for binding more favorable. Macrocyclization is often accomplished by forming an amide bond between the peptide N—and C-termini, between a side chain and the N— or C-terminus [e.g., with K₃Fe(CN)₆ at pH 8.5] (Samson et al., Endocrinology, 137: 5182-5185 (1996)), or between two amino acid side chains. See, e.g. DeGrado, Adv Protein Chem, 39: 51-124 (1988). Disulfide bridges are also introduced into linear sequences to reduce their flexibility. See, e.g. Rose, et al., Adv Protein Chem, 37: 1-109 (1985); Mosberg et al., Biochem Biophys Res Commun, 106: 505-512 (1982). Furthermore, the replacement of cysteine residues with penicillamine (Pen, 3-mercapto-(D) valine) has been used to increase the selectivity of some opioid-receptor interactions. Lipkowski and Carr, Peptides: Synthesis, Structures, and Applications, Gutte, ed., Academic Press pp. 287-320 (1995).

Methods of Decreasing Shiga Toxin and/or Shiga-like Toxin Binding to a Host Cell

Cell surface binding of shiga and/or shiga-like toxin is inhibited (e.g. decreased) by contacting a cell with the SI fusion peptide of the invention. The SI fusion protein sterically inhibits cell surface binding of the bacterial toxin, thereby preventing bacterial toxin infection. Alternatively, cell surface binding of shiga and/or shiga-like toxin is inhibited (e.g., decreased) by contacting shiga and/or shiga-like toxin with the SI fusion peptide of the invention, whereby the SI fusion peptide binds to shiga toxin and/or shiga-like toxin, thereby preventing shiga toxin and/or shiga-like toxin from binding to its natural epitope, thereby preventing bacterial toxin infection. The shiga or shiga-like toxin is for example shiga toxin, shiga-like toxin 1 or shiga-like toxin 2. The shiga-like toxin producing bacteria is, for example, Shigella dysenteriae, enterohaemorrhagic E. coli (EHEC), Aeromononas caviae, Aeromononas hydrophila, Citrobacter freundii and/or Enterobacter cloaca.

Inhibition of attachment is characterized by a decrease in cell internalization and decrease in inhibition of protein synthesis. The SI peptide is contacted with one or more cells of a subject by systemic and/or rectal administration of the SI fusion peptide to the subject. SI peptides are administered in an amount sufficient to decrease (e.g., inhibit) bacterial toxin-cell surface binding and/or internalization. Alternatively, shiga and/or shiga-like toxin producing bacteria are directly contacted with the SI peptide. Shiga/shiga-like toxin cell surface binding is measured using standard immunocytochemical assays known in the art, e.g. by measuring toxin binding to cells using radioactively, or by other means, labeled toxins, by detecting attached toxins using anti-shiga-toxin antibodies, or by measuring protein synthesis levels following toxin-cell contact or exposure.

The methods are useful to alleviate the symptoms of shiga toxin and/or shiga-like toxin infection or a disease associated with a shiga toxin and/or shiga-like toxin. Symptoms associated with shiga toxin and/or shiga-like toxin infection include for example, diarrhea, hemorrhagic colitis and/or hemolytic uremic syndrome.

The methods described herein lead to a reduction in the severity or the alleviation of one or more symptoms of a shiga toxin and/or shiga-like toxin infection or disorder such as those described herein. Shiga toxin and/or shiga-like toxin infection or disorders associated with infection by shiga toxin and/or shiga-like toxin are diagnosed and or monitored, typically by a physician using standard methodologies.

The subject is e.g. any mammal, e.g. a human, a primate, mouse, rat, dog, cat, cow, horse, pig. The treatment is administered prior to bacterial toxin infection or diagnosis of the disorder. Alternatively, treatment is administered after a subject has an infection.

Efficaciousness of treatment is determined in association with any known method for diagnosing or treating the particular bacterial toxin infection or disorder associated with a bacterial toxin infection. Alleviation of one or more symptoms of the bacterial toxin infection or disorder indicates that the compound confers a clinical benefit.

### Pharmaceutical Compositions Including SI Fusion Polypeptides or Nucleic Acids Encoding Same

The SI fusion proteins, or nucleic acid molecules encoding these fusion proteins, (also referred to herein as “Therapeutics” or “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The active agents disclosed herein can also be formulated as liposomes. Liposomes are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an SI fusion protein) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Oral or parenteral compositions are formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g. U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Sustained-release preparations can be prepared, if desired. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The pharmaceutical compositions can be included in a container, pack, or dispenser together with instructions for administration.

The invention will be further illustrated in the following non-limiting examples.

### EXAMPLE 1

### Engineering Stable Cell Lines Secreting IgG Fc Fusions of P-selectin Glycoprotein Ligand-1 Carrying Galα4Galβ3GalNacα and/or a Galα4Galα4GlcNac Glycans

The PSGL-1/mIgG_{2b} expression plasmid is stably transfected into M. Brassicae insect cells having endogenous α1,4 galactosyltransferase activity to produce the desired epitope, Galα4Galβ3GalNAcα. Alternatively, the PSGL-1/mIgG_{2b} expression plasmid is transfected together with α1,4 galactosyltransferase and core 2β1,6-N-acetylglucosaminyltransferase into CHO-K1 cells to produce the Galα4Galβ4GlcNAc (blood group P1 epitope) structure. Alternatively, the PSGL-1/mIgG_{2b} expression plasmid is transfected into E. Coli cells together with α1,4galactosyltransferase, one or more peptide GalNAcTs, and optionally one or more enzymes capable of creating galactose, N-acetylgalactosamine, as well as UDP-Gal and UDP-GalNAc from existing carbohydrate precursors in E. coli cells, to produce the Galα4Galβ4GlcNAc (blood group P1 epitope) structure. Stable clones are selected based on resistance to different selection drugs.

Cell Culture

M. Brassicae cells are cultured in the appropriate selection medium. CHO-K1 cells are cultured in Dulbecco's modified Eagle's medium (DMEM) with 10% fetal bovine serum (FBS) and 25 μg/ml gentamicin sulfate. The selection media contains one or more drug selection factors (e.g., puromycin, hygromycin B, G418 and/or zeocin).

Construction of Expression Plasmids

An α1,4GalT expression plasmid is constructed and a PSGL-1/mIgG_{2b} expression plasmid is constructed as described in Liu et al., Transplantation, 63, 1673 (1997). A core 2β1,6-N-acetylglucosaminyltransferase expression plasmid is constructed as described in Liu et al, Glycobiology, 15(6): 571 (2005).

DNA Transfection and Clonal Selection: M. Brassicae Cells:

M. Brassicae cells are seeded in 75 cm² T-flasks and transfected approximately 24 hours later or when cell confluency reaches 70-80%. Twenty-four hours after transfection, cells in each T-flask are split into several 100 mm petri dishes and incubated in selection medium containing a drug selection factor (e.g., puromycin, hygromycin b, G418 and/or zeocin). The drug resistant clones are formed after approximately two weeks. Clones are identified under the microscope and hand-picked using a pipetman. Selected colonies are cultured in 96-well plates in the presence of selection drugs for another two weeks. Cell culture supernatants are harvested when the cells had reached 80-90% confluency. The concentration of PSGL-1/mIgG_{2b} is assessed by ELISA, SDS-PAGE and/or Western blotting using a goat anti-mouse IgG Fc antibody.

DNA Transfection and Clonal Selection: CHO-K1 Cells

Adherent CHO-K1, cells are seeded in 75 cm² T-flasks and transfected approximately 24 hours later or when cell confluency reaches 70-80%. A modified polyethylenimine (PEI) transfection method may be used for transfection (Boussif, O. et al., 1995; He, Z. et al., 2001). Twenty-four hours after transfection, cells in each T-flask are split into several 100 mm petri dishes and incubated in selection medium containing the one or more drug selection factors (e.g., puromycin, hygromycin b and/or G418). The drug resistant clones are formed after approximately two weeks. Clones are identified under the microscope and hand-picked using a pipetman. Selected colonies are cultured in 96-well plates in the presence of selection drugs for another two weeks. Cell culture supernatants are harvested when the cells had reached 80-90% confluency. The concentration of PSGL-1/mIgG_{2b} is assessed by ELISA, SDS-PAGE and/or Western blotting using a goat anti-mouse IgG Fc antibody. The clones with the highest PSGL-1/mIgG_{2b} expression are transfected with the α1,4GalT encoding plasmid and selected using a different drug selection factor than used to select for PSGL-1/mIgG_{2b} clones. Resistant clones are isolated and characterized by ELISA, SDS-PAGE and Western blot.

Galα4Galβ3GalNAcα and Galα4Galβ4GlcNac Carbohydrate Epitope Density on, and Quantification of, PSGL-1/mIgG_{2b} Using an Enzyme-Linked Immunosorbent Assay

The concentration of recombinant PSGL-1/mIgG_{2b} in cell culture supernatants, and its relative α-Gal epitope density, may be determined by a two-antibody sandwich ELISA as follows. The 96-well ELISA plate is coated overnight at 4° C. with an affinity-purified, polyclonal goat anti-mouse IgG Fc antibody (cat. nr. 55482; Cappel/Organon Teknika, Durham, N.C.) at a concentration of 20 μg/ml. The plate is blocked with 1% BSA in PBS for 1 hour. The supernatant containing PSGL-1/mIgG_{2b} is incubated for 4 hours and then washed three times with PBS containing 0.5% (v/v) Tween 20. After washing, the plate is incubated with a peroxidase-conjugated, anti-mouse IgG Fc antibody (cat.no. A-9917; Sigma) in a 1:3,000 dilution or with peroxidase-conjugated GSA I IB₄-lectin (cat.no. L-5391; Sigma) diluted 1:2,000, for two hours. Bound peroxidase-conjugated antibody or peroxidase-conjugated GSA-lectin is visualized with 3,3′,5,5′-Tetramethylbenzidine dihydrochloride (cat. nr. T-3405; Sigma, Sweden). The reaction is stopped by 2M H₂SO₄ and the plates read at 450 nm. The PSGL-1/mIgG_{2b} concentration is estimated using for calibration a dilution series of purified mouse IgG Fc fragments (cat. Nr. 015-000-008; Jackson ImmunoResearch Labs., Inc., West Grove, Pa.) resuspended in the medium used for fusion protein production or in PBS containing 1% BSA. The epitope density is determined by comparing the relative O.D. from the two ELISAs (GSA-reactivity/anti-mouse IgG reactivity).

### EXAMPLE 2

### Inhibiting Bacterial Toxin Infection In Vitro

Shiga toxin and/or shiga-like toxin and endothelial cells susceptible to the cytopathic effects of the shiga and/or shiga-like toxins are used to assess the inhibitory capacity of the above described fusion proteins with regards to preventing toxin-cell surface binding and disruption of protein synthesis in susceptible host cells.

### EXAMPLE 3

### Routes of Administration

Recombinant PSGL-1/mIgG_{2b} carrying Galα4Galβ3GalNacα and/or a Galα4Galβ4GlcNac glycans (i.e., the STI fusion protein) is administered systemically to prevent haemolytic uremic syndrome. The STI fusion protein is administered rectally to prevent spreading from the site of infection.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A fusion polypeptide comprising a first polypeptide linked to a second polypeptide, wherein the first polypeptide is a mucin polypeptide glycosylated by an α1,4galactosyltransferase, and the second polypeptide comprises at least a region of an immunoglobulin polypeptide.

2. The fusion polypeptide of claim 1, wherein said mucin polypeptide is further glycosylated by a core 2β1,6-N-acetylglucosaminyltransferase.

3 . The fusion polypeptide of claim 1 or 2, wherein said mucin polypeptide has a glycan repertoire including a Galα4Galβ3GalNacα structure or a Galα4Galβ4GlcNac structure.

4. The fusion polypetide of claim 1, wherein said mucin polypeptide is selected from the group consisting of PSGL-1, MUC1, MUC2, MUC3, MUC4, MUC5a, MUC5b, MUC5c, MUC6, MUC11, MUC12, CD34, CD43, CD45, CD96, GlyCAM-1, and MAdCAM-1 or fragment thereof.

5. The fusion polypeptide of claim 4, wherein said mucin polypeptide comprises at least a region of a P-selectin glycoprotein ligand-1 (PSGL-1).

6. The fusion polypeptide of claim 5, wherein said mucin polypeptide includes an extracellular portion of a P-selectin glycoprotein ligand-1.

7. The fusion polypeptide of claim 1, wherein the second polypeptide comprises a region of a heavy chain immunoglobulin polypeptide.

8. The fusion polypeptide of claim 1, wherein said second polypeptide comprises an Fc region of an immunoglobulin heavy chain.

9. A method for preventing or alleviating a symptom of bacterial toxin infection in a subject in need thereof, the method comprising administering to the subject fusion polypeptide of claim 1.

10. The method of claim 9, wherein said fusion polypeptide is administered to the subject systemically.

11. The method of claim 9, wherein said fusion polypeptide is administered to the subject rectally.

12. The method of claim 9, wherein said bacterial toxin is produced by a bacteria selected from the group consisting of Shigella dysenteria, enterohaemorrhagic E. coli, Aeromononas caviae, Aeromononas hydrophila, Citrobacter freundii, and Enterobacter cloacae.

13. The method of claim 12, wherein the bacterial toxin is Shiga toxin or Shiga-like toxin 1.

14. The method of claim 12, wherein the bacterial toxin is Shiga-like toxin 2.

15. A method of producing a mucin-immunoglobulin fusion polypeptide comprising:
a) providing a cell comprising:
i) a nucleic acid encoding a mucin polypeptide linked to a nucleic acid encoding at least a portion of an immunoglobulin polypeptide;
ii) a nucleic acid encoding a α1,4galactosyltransferase polypeptide; and
iii) optionally a nucleic acid encoding a core 2β1,6-N-acetylglucosaminyltransferase; and
b) culturing the cell under conditions that permit production of said mucin-immunoglobulin fusion polypeptide wherein said fusion polypeptide has a glycan repertoire including a Galα4Galβ3GalNAc α structure or a Galα4Galβ4GlcNac structure; and
c) isolating said mucin-immunoglobulin fusion polypeptide.

16. The method of claim 15, wherein said cell is a eukaryotic cell or a prokaryotic cell.

17. The method of claim 16, wherein said eukaryotic cell is a mammalian cell, or a yeast cell.

18. The method of claim 17, wherein said mammalian cell is a CHO cell.

19. The method of claim 16, wherein said prokaryotic cell is a bacterial cell.

20. A cell comprising:
a) a nucleic acid encoding a mucin polypeptide linked to a nucleic acid encoding at least a portion of an immunoglobulin polypeptide;
b) a nucleic acid encoding a α1,4galactosyltransferase polypeptide; and
c) optionally a nucleic acid encoding a core 2β1,6-N-acetylglucosaminyltransferase.


SEQUENCE LISTING (2 sequences)

SEQ ID NO: 1 - protein, 10 aa, Artificial sequence
  PSGL-1 consensus sequence
XQTTXXXXXE

SEQ ID NO: 2 - protein, 10 aa, Artificial sequence
  PSGL-1 consensus sequence
AXTTXXAXXE